(19) United States
(12) Patent Application Publication
(10) Pub. No.: US 2007/0118161 A1
(43) Pub. Date: May 24, 2007

(54) Non-snag polymer ligating clip

(76) Inventors: Daniel L. Kennedy, Wake Forest, NC (US); Donald F. Wilson JR., Raleigh, NC (US); Bryan D Knodel, Flagstaff, AZ (US)

Correspondence Address: BAKER & HOSTETLER LLP, WASHINGTON SQUARE, SUITE 1100, 1050 CONNECTICUT AVE. N.W., WASHINGTON, DC, 20036-5304, US

(21) Appl. No.: 11/283,848

(22) Filed: Nov. 22, 2005

(51) Int. Cl. A61B 17/08

(52) U.S. Cl. 606/157

(57) ABSTRACT

A polymeric, surgical clip having first and second curved legs with each having a pair of opposing side surfaces joined at their proximal ends by a flexible hinge section and movable from an open position to a closed position for clamping a vessel between curved opposing inner surfaces. The first leg terminates at its distal end in a female locking member, and the second leg member terminates in a male locking member complimentary to the female locking member such that when the first and second leg members are moved from an open position to a closed position about the hinge section the male member is lockingly engaged in the female locking member. The clip is provided with low profile boss-like elements on the legs thereof to reduce the risk of snagging a suture during coronary artery bypass graft surgery.

### TECHNICAL FIELD

The present invention relates to surgical clips, and more particularly to a non-snag polymer ligating clip which is easier to introduce into tight spaces during coronary artery bypass graft surgery and which is less prone to catch a suture when attaching a graft IMA (internal mammary artery) to the coronary artery during the coronary artery bypass graft procedure. More particularly, the present disclosure relates to an improved surgical ligating clip that can be used in coronary artery bypass graft surgical procedures without catching a suture during attachment of a graft IMA to the coronary artery and which subsequently allows for CT (computer tomography) to check patency in the coronary artery bypass graft juncture since the clip is translucent and not visible on CT.

### BACKGROUND ART

Many surgical procedures require vessels or other tissues of the human body to be ligated during the surgical process. For example, many surgical procedures require cutting blood vessels (e.g., veins or arteries), and these blood vessels may require ligation to reduce bleeding. In some instances, a surgeon may wish to ligate the vessel temporarily to reduce blood flow to the surgical site during the surgical procedure. In other instances a surgeon may wish to permanently ligate a vessel. Ligation of vessels or other tissues can be performed by closing the vessel with a ligating clip, or by suturing the vessel with surgical thread. The use of surgical thread for ligation requires complex manipulations of the needle and suture material to form the knots required to secure the vessel. Such complex manipulations are time-consuming and difficult to perform, particularly in endoscopic surgical procedures, which are characterized by limited space and visibility. By contrast, ligating clips are relatively easy and quick to apply. Typically, a clip is applied to the vessel or other tissue by using a dedicated mechanical instrument commonly referred to as a surgical clip applier, ligating clip applier, or hemostatic clip applier. Accordingly, the use of ligating clips in endoscopic as well as open surgical procedures has grown dramatically.

Ligating clips can be classified according to their geometric configuration (e.g., symmetric clips or asymmetric clips), and according to the material from which they are manufactured (e.g., metal clips or polymeric clips). Symmetric clips are generally “U” or “V” shaped and thus are substantially symmetrical about a central, longitudinal axis extending between the legs of the clip. Symmetric clips are usually constructed from metals such as stainless steel, titanium, tantalum, or alloys thereof. By means of a dedicated clip applier, the metal clip is permanently deformed over the vessel. An example of one such clip is disclosed in U.S. Pat. No. 5,509,920 to Phillips et al. An example of a metallic clip applier is disclosed in U.S. Pat. No. 3,326,216 to Wood in which a forceps-type applier having conformal jaws is used to grip and maintain alignment of the clip during deformation. Such appliers may additionally dispense a plurality of clips for sequential application, as disclosed in U.S. Pat. No. 4,509,518 to McGarry et al.

With the advent of high technology diagnostic techniques using computer tomography (CATSCAN or CT) and magnetic resonance imaging (MRI), metallic clips have been found to interfere with the imaging techniques. To overcome such interference limitations, biocompatible polymers have been increasingly used for surgical clips. Unlike metallic clips, which are usually symmetric, polymeric clips are usually asymmetric in design and hence lack an axis of symmetry. Inasmuch as the plastic clip cannot be permanently deformed for secure closure around a vessel or other tissue, latching mechanisms have been incorporated into the clip design to establish closure conditions and to secure against re-opening of the vessel. For example, well known polymeric clips are disclosed in U.S. Pat. No. 4,834,096 to Oh et al. and U.S. Pat. No. 5,062,846 to Oh et al., both of which are assigned to the assignee of the presently disclosed subject matter. These plastic clips generally comprise a pair of curved legs joined at their proximal ends with an integral hinge or heel. The distal ends of the curved legs include interlocking latching members. For example, the distal end of one leg terminates in a lip or hook structure into which the distal end of the other leg securely fits to lock the clip in place.

The distal ends of the clips taught in U.S. Pat. No. 5,062,846 to Oh et al. also include lateral bosses that are engaged by the jaws of a clip applier. A clip applier specifically designed for asymmetric plastic clips is used to close the clip around the tissue to be ligated, and to latch or lock the clip in the closed condition. In operation, the jaws of this clip applier are actuated into compressing contact with the legs of the clip. This causes the legs to pivot inwardly about the hinge, thereby deflecting the hook of the one leg to allow reception therein of the distal end of the other leg. A clip applier designed for use with asymmetric plastic clips in an open (i.e., non-endoscopic) surgical procedure is disclosed in U.S. Pat. No. 5,100,416 to Oh et al., also assigned to the assignee of the presently disclosed subject matter.

In addition to compatibility with sophisticated diagnostic techniques, asymmetric clips have other advantages over symmetric clips. For example, because asymmetric clips are formed from polymeric materials, the mouths of asymmetric clips can generally be opened wider than the mouths of symmetric clips. This allows a surgeon to position the clip about the desired vessel with greater accuracy. In addition, a clip of the type described in the aforementioned U.S. Pat. Nos. 4,834,096 and 5,062,846 can be repositioned before locking the clip on the vessel or before removing the clip from the vessel, in a process referred to as “approximating” the clip.

Various types of hemostatic and aneurysm asymmetric clips are used in surgery for ligating blood vessels or other tissues to stop the flow of blood. Such clips have also been used for interrupting or occluding ducts and vessels in particular surgeries such as sterilization procedures.

As is well known to those skilled in the art, metal ligating clips are traditionally used to tie off the branches of an IMA being used for the graft in a coronary artery bypass graft procedure. However, metal clips are not viable when there is a desire to use a CT scan to study the patency of a graft after bypass surgery. The use of the CT scan is desirable since it eliminates the need for re-catheterization in order to verify graft patency.

Thus, there is a long-felt need in coronary artery bypass surgery to find an alternative to the metal ligating clip used to tie off branches of the IMA being used for a coronary artery bypass graft since the metal ligating clips do not readily lend themselves to CT scans to study the patency of a graft after a bypass. Thus, a re-catheterization is many times necessary in order to verify the patency. The radiolucent polymeric ligating clip such as the HEM-O-LOK® available from Pilling Weck would be ideal except for the side bosses which facilitate engagement and application of the clip by a clip applier. The applicant has now discovered a novel modification to the polymeric ligating clip such that the clip will provide all of the advantages of a traditional polymeric clip including radiolucency but will not possess the disadvantages of the suture-snagging bosses provided on prior art polymeric ligating clips such as the HEM-O-LOK® clip. The new and improved non-snag polymeric ligating clip provides a low profile clip that is ideal for coronary artery bypass graft surgery.

### SUMMARY

In accordance with the present disclosure, a polymeric surgical clip is provided of the type comprising first and second legs joined at their proximal ends by a flexible hinge section. Each leg has a vessel clamping inner surface, an opposite outer surface, and a pair of opposing side surfaces. The vessel clamping inner surface is in opposition to the vessel clamping inner surface of the other leg. Further, a female locking member is positioned on the distal end of the first leg and a male locking member is positioned on the distal end of the second leg. The female and male locking members are formed such that when the first and second leg members are moved from an open position to a closed position about the hinge section, the male locking member is lockingly engaged in the female locking member so as to removably lock the clip in the closed position.

Further in the preferred embodiment, the inner vessel-clamping surface of the first leg has a concave radius of curvature and the outer surface has a convex radius of curvature between the hinge section and the distal end. In the same embodiment, the inner vessel-clamping surface of the second leg has a convex radius of curvature and the outer surface has a concave radius of curvature between the hinge section and the distal end.

Still further in the preferred embodiment, a first pair of recesses are formed in opposite sides of the first leg between the hinge section and the distal end of the first leg, and a second pair of recesses are formed in opposite sides of the second leg adjacent the distal end of the second leg, such that the first and second pair of recesses serve to allow for engagement and application of the surgical clip by a clip applier apparatus.

In another embodiment of the non-snag polymer ligating clip, a first recess is located on the outer surface of the first leg between the hinge section and the distal end of the first leg, and a second recess is located in the outer surface of the second leg adjacent the distal end of the second leg such that the first and second recesses serve to allow for engagement and application of the surgical clip by a clip applier apparatus.

In another embodiment of the non-snag polymer ligating clip, a first protuberance is located on the outer surface of the first leg between the hinge section and the distal end of the first leg, and a second protuberance is located on the outer surface of the second leg adjacent the distal end of the second leg such that the first and second protuberances serve to allow for engagement and application of the surgical clip by a clip applier apparatus.

In still another embodiment of the non-snag polymer ligating clip of the discovery, a first pair of arcuate protuberances are joined to opposite sides of the first leg between the hinge section and the distal end of the first leg, and a second pair of arcuate protuberances are joined to opposite sides of the second leg adjacent the distal end of the second leg such that the first and second pair of arcuate protuberances serve to allow for engagement and application of the surgical clip by a clip applier apparatus.

The non-snag polymeric surgical clip disclosed herein is most suitably made of polymeric material and accordingly minimizes interference with high technology diagnostic modalities such as CATSCAN, MRI and MRS. At the same time, the clip is nearly as small as comparable metal clips while maintaining sufficient strength and possessing a high degree of security in the clip's latching mechanism. The ligating clip of the discovery is further configured with low profile bosses used for engagement and application by a clip applier apparatus, and wherein the low profile bosses will facilitate introduction into tight spaces during coronary artery bypass graft procedures and very importantly is less prone to snag a suture during coronary artery bypass graft surgical procedures than a conventional polymeric ligating clip having conventional bosses at the end of both the first and second leg that extend outwardly from the side surfaces thereof.

It is therefore an object of the presently disclosed non-snag surgical ligating clip to provide a non-snag polymeric surgical clip that is particularly well suited for coronary artery bypass graft surgical procedures.

Some of the objects of the subject matter disclosed herein having been stated hereinabove, other objects will become evident as the description proceeds when taken in connection with the accompanying drawings as best described hereinbelow.

### BRIEF DESCRIPTION OF THE DRAWINGS

FIG. 1 is a perspective view of a prior art polymeric ligating clip;

FIG. 2 is another perspective view of the prior art ligating clip shown in FIG. 1;

FIG. 3 is a perspective view of one embodiment of the polymeric ligating clip of the present discovery;

FIG. 4 is a perspective view of another embodiment of the polymeric ligating clip of the present discovery;

FIG. 5 is a perspective view of another embodiment of the polymeric ligating clip of the present discovery;

FIG. 6 is a perspective view of another embodiment of the polymeric ligating clip of the present discovery;

FIG. 7 is a perspective view of another embodiment of the polymeric ligating clip of the present discovery;

FIG. 8 is a perspective view of still another embodiment of the polymeric ligating clip of the present discovery; and

FIG. 9 is a perspective view of still another embodiment of the polymeric ligating clip of the present discovery.

### DETAILED DESCRIPTION

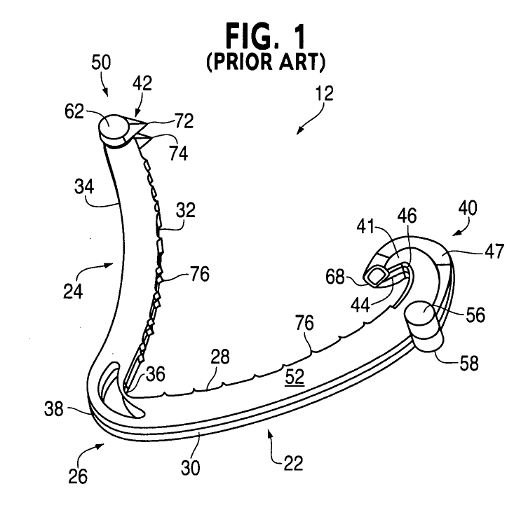

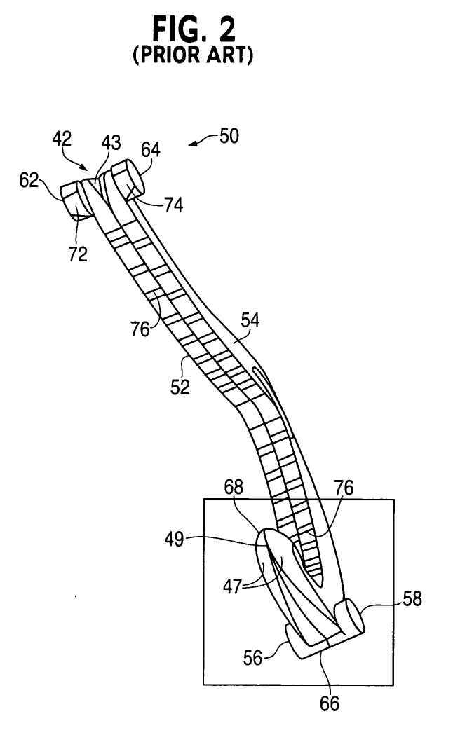

Referring first to FIGS. 1-2 of the drawings, one example is illustrated of a conventional asymmetric surgical clip 12. Clip 12 and others of similar design are particularly useful as hemostatic clips that can be latched around a vessel or other type of tissue to ligate the vessel and thereby stop or reduce the flow of fluid through the vessel. Clip 12 can be constructed from any suitable biocompatible material. However, the presently disclosed subject matter is particularly suitable for practice with polymeric clips. Thus, clip 12 preferably comprises a one-piece integral polymeric body formed from a suitable strong biocompatible engineering plastic such as the type commonly used for surgical implants. Examples include, but are not limited to, acetyl polyoxym ethylene (POM), polyethylene terephthalate (PET), polybutylene terephthalate (PBT), polyoxymethylene, or other thermoplastic materials having similar properties that can be injection-molded, extruded or otherwise processed into like articles.

Now turning to FIG. 1, the body of clip 12 comprises a first or outer leg, generally designated 22, and a second or inner leg, generally designated 24. First and second legs 22 and 24 are joined at their proximal ends by an integral hinge section, generally designated 26. First and second legs 22 and 24 each have a pair of opposing side surfaces 52 and 54. First and second legs 22 and 24 also have complementary arcuate profiles. Thus, first leg 22 has a concave inner surface 28 and a convex outer surface 30, and second leg 24 has a convex inner surface 32 and a concave outer surface 34. Convex inner surface 32 of second leg 24 and concave inner surface 28 of first leg 22 have substantially matching radii of curvature.

Hinge section 26 has a continuous concave inner surface 36 and a continuous convex outer surface 38. Concave inner surface 36 of hinge section 26 joins concave inner surface 28 of first leg 22 and convex inner surface 32 of second leg 24. Convex outer surface 38 of hinge section 26 joins convex outer surface 30 of first leg 22 and concave outer surface 34 of second leg 24.

First leg 22 terminates in a female locking member 40 at its distal end. Female locking member 40 comprises a resilient inwardly turned hook 41. Second leg 24 terminates in a male locking member 50. Male locking member 50 comprises a pointed tip section 42 at its distal end. Hook 41 is distally curved inwardly toward hinge section 26, and has a transverse beveled surface 44. Beveled surface 44 and concave inner surface 28 define a latching recess 46, which is adapted for conformally engaging tip section 42 of male locking member 50 in the course of compressing clip 12 into a latched or locked position around a vessel or other tissue.

As best shown in FIG. 2, the top surface of hook 41 most preferably comprises two convex surfaces 47 that come together to define a sharp crest-like cutting edge 49 to facilitate cutting through connective tissue adjacent a vessel or other tissue during latching of the clip therearound. However, this is not a required feature of clip 12.

As best shown in FIG. 2, clip 12 comprises opposing side surfaces 52 and 54. Typically, the body of clip 12 has a constant thickness between side surfaces 52 and 54. Adjacent to the distal end of the first leg 22 and immediately inwardly of hook 41, a pair of cylindrical bosses 56 and 58 are formed coaxially on the opposed side surfaces 52 and 54, respectively, of first leg 22. In the illustrated example of clip 12, a bridge section 66 couples bosses 56 and 58 together. As evident in FIG. 1, bosses 56 and 58 project outwardly beyond convex outer surface 30 of first leg 22.

Referring again to the distal end of second or inner leg 24, another pair of cylindrical bosses 62 and 64 is formed coaxially on the opposed lateral surfaces of inner leg 24 at tip section 42. As evident in FIGS. 1 and 2, bosses 62 and 64 of second leg 24 extend longitudinally forward beyond tip section 42.

Also, as best shown in FIGS. 1 and 2, hook 41 of first leg 22 preferably terminates at a sharp tip 68 with cutting edge 49 extending at least along a portion of the length of the top surface of hook 41. Male locking member 50 of second leg 24 includes a pair of inwardly directed sharp tissue-penetrating teeth 72 and 74, to assist in gripping, stretching and piercing nearby connective tissue, in concert with cutting edge 49 and sharp tip 68 on hook 41.

Both first and second legs 22 and 24 have a plurality of optional protrusions or teeth 76 extending from their respective inner surfaces 28 and 32. These features are designed to engage the tissue of the vessel being clamped and assist in preventing the vessel from sliding laterally or longitudinally during or following clip closure. It will be noted, however, that other clips equally suitable for use in conjunction with the presently disclosed subject matter may not contain such features.

### Conventional Clip Closure

In the practice of ligating and cutting a vessel or other tissue, as understood by persons skilled in the art, clip 12 is designed to be compressed into a latched or locked closed position around the vessel through the use of an appropriate clip applicator instrument. The clip applicator instrument engages protruding bosses 56, 58, 62 and 64 of clip 12 and pivots bosses 56, 58, 62 and 64 inwardly about hinge section 26. This causes first and second legs 22 and 24 to close around the vessel, with convex inner surface 32 of second leg 24 and complementary concave inner surface 28 of first leg 22 contacting the outer wall of the vessel.

However, before any contact is made between first and second legs 22 and 24, sharp tissue penetrating teeth 72 and 74 on bosses 62 and 64 of second leg 24 start to indent and penetrate any connective tissue surrounding the vessel therebetween and pull the tissue down. Simultaneously, sharp tip 68 and hook 41 on first leg 22, while sliding between teeth 72 and 74, also begin to penetrate the tissue and force the tissue up. Sharp tip 68 and cutting edge 49 on hook 41 enter a groove 43 of pointed tip section 42 on second leg 24, thereby beginning puncturing and cutting of the connective tissue.

As cutting edge 49 and sharp tip 68 of hook 41 continue to move through groove 43 between teeth 72 and 74, shear forces contribute to further puncturing and cutting of the connective tissue surrounding the vessel. If all the tissue is still not cut between the distal portion of second leg 24 and hook 41, it will stretch and become thinner until it is easily punctured by sharp tip 68 and cut by cutting edge 49 of hook 41 as it passes through groove 43 of second leg 24. Once the connective tissue is cut, female and male locking members 40 and 50 are able to lockingly engage without interference.

It should be understood that while cutting edge 49 is a desired feature of the preferred embodiment of clip 12, other embodiments of clip 12 that do not include cutting edge 49 are contemplated to be part of the prior art and clip 12. Thus, clip 12 may or may not include cutting edge 49 as described above.

Tip section 42 of second leg 24 then begins to contact female locking member 40 at hook 41. Further pivotal movement by the jaws of the applicator instrument longitudinally elongates first leg 22 and deflects hook 41, allowing tip section 42 of male locking member 50 to align with latching recess 46 of female locking member 40. Upon release of the applicator instrument, tip section 42 snaps into and is conformably seated in latching recess 46 of female locking member 40, at which point clip 12 is in its latched and closed position. In the latched condition, tip section 42 is engaged between concave inner surface 28 and beveled surface 44, thereby securely clamping a designated vessel or other tissue between concave inner surface 28 and convex inner surface 32. After clip 12 is secured in its closed position and a vessel is ligated, most likely with two clips 12 on either side of the cutting site, the physician can safely cut the vessel.

### NON-SNAG CLIP EMBODIMENT

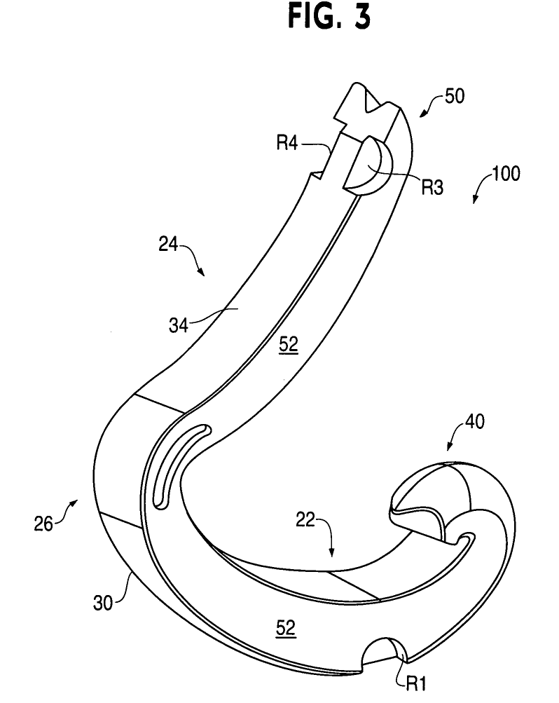

It would be desirable to use polymeric-type ligating clips similar to clip 12 shown in FIGS. 1 and 2 in coronary artery bypass graft surgical procedures in lieu of metal clips conventionally used for this purpose. Metal ligating clips have a significant limitation since they do not allow for a CT scan and thus a surgeon must many times re-catheterize to verify patency of grafts after a bypass using CT scanning. Conventional polymeric ligating clips 12 are problematic since when attaching a graft IMA to the coronary artery, a running stitch must be used to join the arteries together. The bosses on the sides of clip 12 present problems to the surgeon since they (1) tend to catch a suture during the attachment procedure and (2) are bulky and awkward to apply in tight spaces during the coronary artery bypass graft procedure. Thus, the novel and improved low profile polymeric ligating clip as shown in FIGS. 3-9 provides the ability to use a polymeric ligating clip in a coronary artery bypass graft procedure without awkwardness in tight surgical spaces and the tendency to snag the suture during attachment of a graft IMA to the coronary artery. The improved low profile polymeric clip described herein is extremely advantageous since its use will allow for CT scanning after a bypass procedure and eliminate the need for re-catheterization to verify patency of grafts after the bypass procedure.

Referring now to FIGS. 3-9 of the drawings, several embodiments of the non-snag polymer ligating clip of the discovery will now be described in detail.

FIG. 3 depicts non-snag polymer ligating clip 100 in accordance with the discovery wherein bosses 56, 58 and 62, 64 (see FIGS. 1 and 2) of conventional clip 12 have been eliminated, and arcuate recesses R1, R2 (not shown, positioned on outer side surface 54 opposite side 52) are provided in opposing sides 52, 54 of first leg 22 between the hinge section 26 and the distal end of the leg comprising female locking member 40. A second pair of recesses R3, R4 are provided in opposite sides 52, 54 of second leg 24 at the distal end adjacent male locking member 50. Applicants note that the same numerals have been designated in FIGS. 3-9 to designate the same elements on the embodiments of the non-snag clip as already designated on representative conventional clip 12 shown in FIGS. 1 and 2. This is intended to facilitate a easier description of the novel embodiments of the new discovery and better understanding thereof from the detailed disclosure herein, but is not intended to limit the features of the present discovery to those of conventional clip 12 shown in FIGS. 1 and 2.

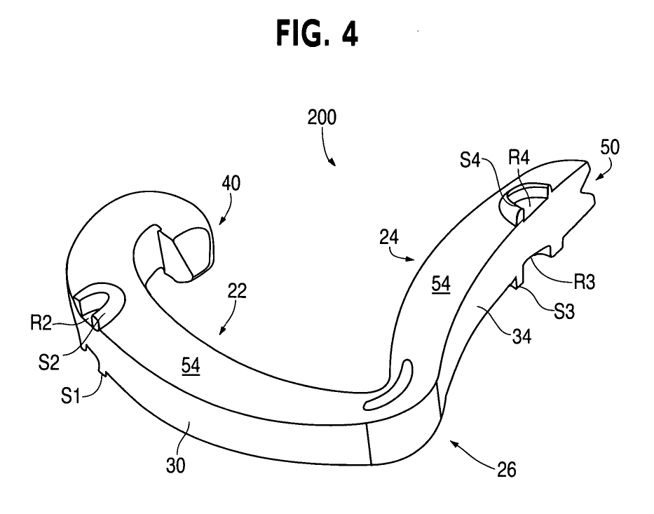

FIG. 4 depicts another embodiment of the non-snag polymer ligating clip generally designated 200 wherein recesses R1, R2 are provided on opposing sides 52, 54 that are adjacent female locking member 40 of first leg 22 and recesses R3, R4 are provided on opposing sides 52, 54 that are adjacent male locking member 50 at the end of second leg 24. Recesses R1, R2 provided on opposing sides 52, 54 of first leg 22 and recesses R3, R4 provided on opposing sides 52, 54 of second leg 24 are each provided with an arcuate, outwardly protruding shroud S1, S2 and S3, S4, respectively, around the recesses thereof.

It will be appreciated that the recesses R1, R2 and R3, R4 in both clips 100 and 200 shown in FIGS. 3 and 4, respectively, are provided to allow a clip applier to engage and apply the non-snag polymer ligating clips of the discovery similarly to the protruding cylindrical bosses 56, 58 and 62, 64 provided in conventional clip 12 shown in FIGS. 1 and 2. However, recesses R1, R2 and R3, R4 are recessed so as not to snag a suture during the coronary artery bypass graft surgical procedure or to impede positioning in the tight spaces available in coronary artery bypass graft procedures.

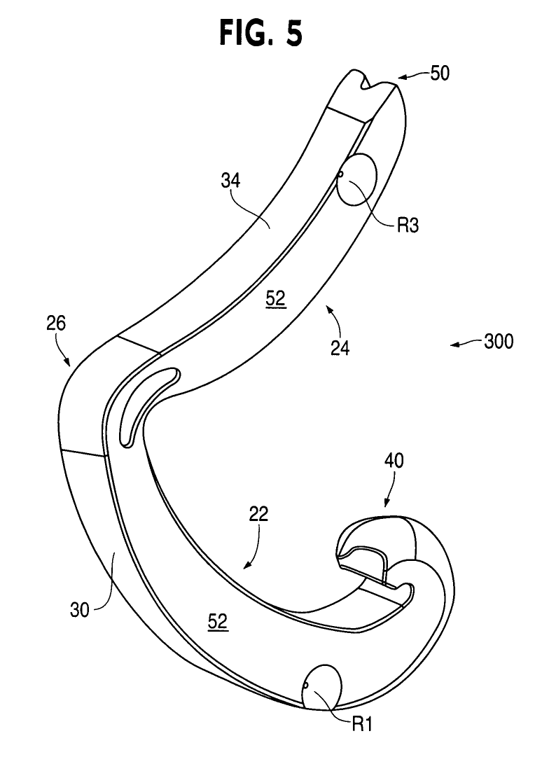

FIG. 5 shows another embodiment of the non-snag polymeric clip, generally designated 300. Clip 300 comprises cone-shaped recesses R1, R2 on opposing sides 52, 54 of first leg 22 adjacent female locking member 40 and cone-shaped recesses R3, R4 on opposing sides 52, 54 of second leg 24 adjacent male locking member 50. Recesses R1, R2 and R3, R4 are intended to provide for engagement and application of clip 300 by a suitable clip engagement and applicator apparatus without the shortcomings of bosses 56, 58 and 62, 64 of conventional polymeric clip 12.

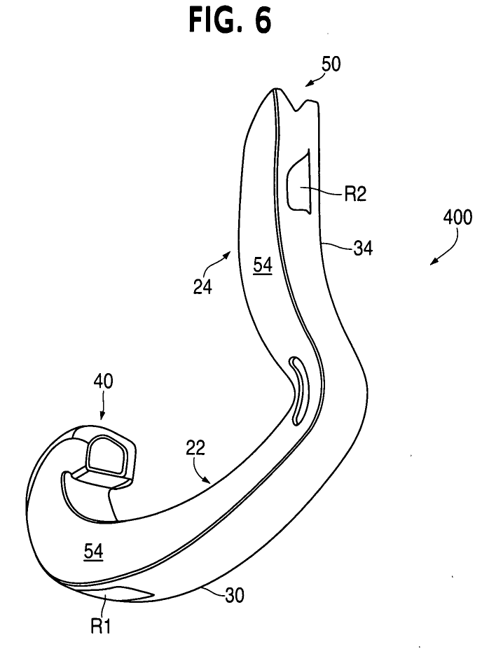

FIG. 6 shows another embodiment of the non-snag polymeric clip of the discovery which is generally designated 400. Clip 400 comprises a first recess R1, in the bottom surface 30 of first leg 22 adjacent female locking member 40 and a second recess R2, defined in the bottom surface 34 of second leg 24 adjacent male locking member 50. Recesses R1, R2 maybe of any suitable lengthwise profile including arcuate (concave or convex) or rectangular profile. Recesses R1, R2 are intended to provide for engagement and application by a suitable clip applicator apparatus (not shown) while not presenting any suture snag danger during use in a coronary artery bypass graft procedure.

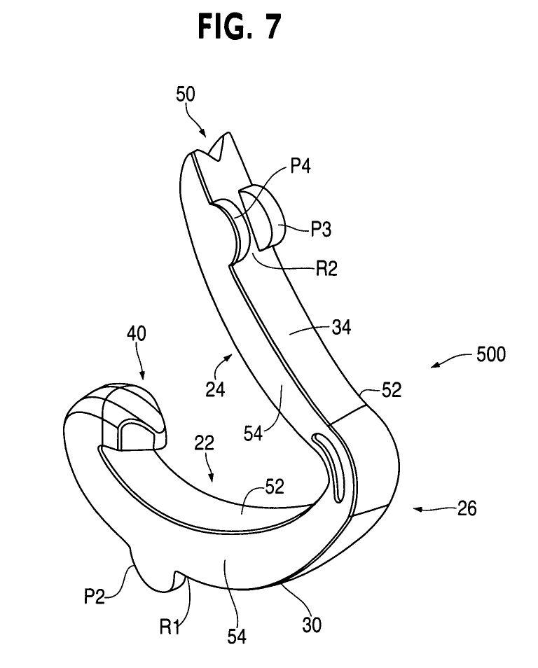

Referring now to FIG. 7, another embodiment of the improved clip of the discovery is shown and generally designated 500. Clip 500 is similar to clip 400 and comprises a first bottom surface recess R1 adjacent the end of first leg 22 and a second bottom surface recess R2 adjacent the end of second leg 24. Recess R1 is defined in the outer surface of leg 22 and recess R2 is defined in the outer surface of leg 24. Clip 500 further comprises a pair of arcuate protuberances P1, P2 extending outwardly from the bottom surface 30 of first leg 22 adjacent each side of recess R1 (P1 not shown, but positioned directly opposite P2 across R1), and a second pair of protuberances P3, P4 extending outwardly from the outer surface 34 of second leg 24 adjacent each side of recess R2. Protuberances P1, P2 are coplanar with side walls 52, 54 of first leg 22 and protuberances P3, P4 are coplanar with side walls 52, 54 of second leg 24. Recess Ri with associated protuberances P1, P2 and recess R2 with associated protuberances P3, P4 are intended to facilitate engagement and application of clip 500 by a clip applicator apparatus (not shown) while not presenting any suture snag danger during use in a coronary artery bypass graft procedure.

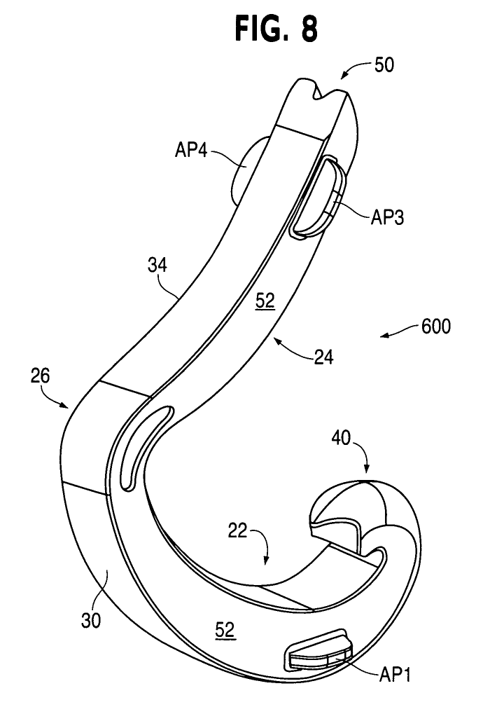

Referring now to FIG. 8, another embodiment of the non-snag polymeric clip of the discovery is shown and generally designated 600. Clip 600 comprises a pair of arcuate protuberances AP1, AP2 (not shown, positioned directly opposite AP1 on opposing face of surface 52) provided on opposing sides 52, 54 of first leg 22 and a pair of opposing arcuate protuberances AP3, AP4 provided on opposing sides 52, 54 of second leg 24. The arcuate protuberances AP1, AP2 and AP3, AP4 are relatively small relative to female locking member 40 and male locking member 50, respectively, and define a substantially rounded, semicircular shape so as to deflect and not catch a suture during the coronary artery bypass graft surgical procedure. Although arcuate protuberances AP1, AP2 on first leg 22 and arcuate protuberances AP3, AP4 on second leg 24 do extend outwardly therefrom, it should be emphasized again that they are relatively small and arcuately shaped so as not to either catch a suture or to be obtrusive in tight spaces during the coronary artery bypass graft surgical procedure.

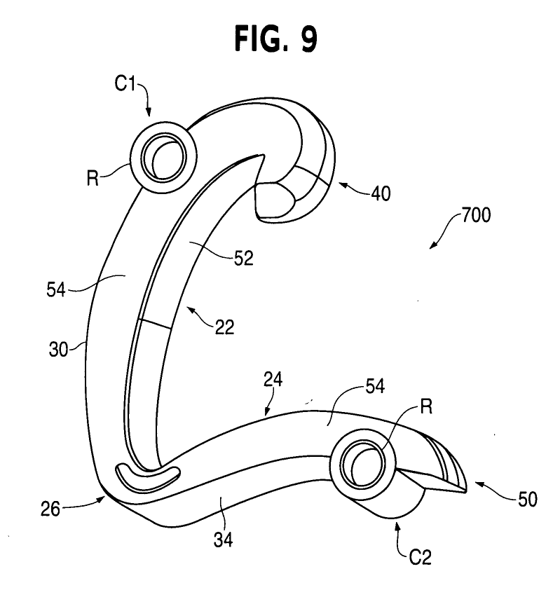

Finally, FIG. 9 shows still another embodiment of the non-snag polymeric surgical clip of the discovery which is generally designated 700. Clip 700 is somewhat similar to clip 200 shown in FIG. 4 and comprises a first cylinder C1 extending across the outer surface 30 of first leg 22 adjacent female locking member 40, and a second cylinder C2 extending across the back surface 34 of second leg 24 adjacent male locking member 50. Cylinder C1 provides an arcuate surface across the outer surface 30 of first leg 22 and further comprises a rounded cylindrical rim R at each end thereof. Cylinder C2 at the end of second leg 24 also provides an arcuate surface across the outer surface 34 of second leg 24. Cylinder C2 comprises a rounded cylindrical rim R on each side thereof similar to cylinder C1 in order to prevent snagging of a surgical suture. Cylinders C1 and C2 extend lengthwise only slightly beyond the side surfaces 52, 54 of first leg 22 and only slightly beyond the side surfaces 52, 54 of first leg 22 and only slightly beyond the side surfaces 52, 54 of second leg 24.

It will be understood that various details of the presently disclosed subject matter can be changed without departing from the scope of the disclosure. Furthermore, the foregoing description is for the purpose of illustration only, and not for the purpose of limitation—the invention being defined by the claims.

What is claimed is:

1. A polymeric non-snag, low profile surgical clip comprising:
(a) a first leg and a second leg, each of said legs having an inner vessel-clamping surface, an outer surface and a pair of opposing side surfaces flanking said inner and outer surfaces, said inner surfaces being positioned in opposition to each other;
(b) a flexible hinge section integrally disposed between and joining the proximal ends of said first and second legs;
(c) a female locking member positioned on the distal end of said first leg and a male locking member positioned on the distal end of said second leg, said female and male locking members being formed whereby when said first and second leg members are moved from an open position to a closed position about said hinge section, said male locking member is lockingly engaged in said female locking member so as to removably lock said first and second leg members of said surgical clip in said closed position; and
(d) a first pair of recesses formed in opposite sides of said first leg between said hinge section and the distal end of said first leg, and a second pair of recesses formed in opposite sides of said second leg adjacent the distal end of said second leg, said first and second pair of recesses serving to allow for engagement and application of the surgical clip by a clip applier apparatus.

2. The surgical clip of claim 1, wherein said inner surface of said first leg has a concave radius of curvature between said hinge section and its distal end, said outer surface of said first leg has a convex radius of curvature between said hinge section and its distal end, said inner surface of said second leg has a convex radius of curvature between said hinge section and its distal end, and said outer surface of said second leg has a concave radius of curvature between said hinge section and its distal end.

3. The surgical clip of claim 1, wherein said hinge section has a continuous concave inner surface and a continuous convex outer surface.

4. The surgical clip of claim 1, wherein said female locking member comprises a resilient inwardly turned hook curved toward said second leg member.

5. The surgical clip of claim 4, wherein said male locking member is complementary to said hook of said female locking member whereby when said first and second leg members are moved from an open position to a closed position about said hinge section, said resilient hook of said female locking member contacts said male locking member and is urged open to receive said male locking member so as to removably lock said first and second leg members of said surgical clip in said closed position.

6. A polymeric non-snag, low profile surgical clip comprising:
(a) a first leg and a second leg, each of said legs having an inner vessel-clamping surface, an outer surface and a pair of opposing side surfaces flanking said inner and outer surfaces, said inner surfaces being positioned in opposition to each other;
(b) a flexible hinge section integrally disposed between and joining the proximal ends of said first and second legs;
(c) a female locking member positioned on the distal end of said first leg and a male locking member positioned on the distal end of said second leg, said female and male locking members being formed whereby when said first and second leg members are moved from an open position to a closed position about said hinge section, said male locking member is lockingly engaged in said female locking member so as to removably lock said first and second leg members of said surgical clip in said closed position; and
(d) a first recess located in the outer surface of said first leg between said hinge section and the distal end of said first leg, and a second recess located in the outer surface of said second leg adjacent the distal end of said second leg, said first and second recesses serving to allow for engagement and application of the surgical clip by a clip applier apparatus.

7. The surgical clip of claim 6, wherein said inner surface of said first leg has a concave radius of curvature between said hinge section and its distal end, said outer surface of said first leg has a convex radius of curvature between said hinge section and its distal end, said inner surface of said second leg has a convex radius of curvature between said hinge section and its distal end, and said outer surface of said second leg has a concave radius of curvature between said hinge section and its distal end.

8. The surgical clip of claim 6, wherein said hinge section has a continuous concave inner surface and a continuous convex outer surface.

9. The surgical clip of claim 6, wherein said female locking member comprises a resilient inwardly turned hook curved toward said second leg member.

10. The surgical clip of claim 9, wherein said male locking member is complementary to said hook of said female locking member whereby when said first and second leg members are moved from an open position to a closed position about said hinge section, said resilient hook of said female locking member contacts said male locking member and is urged open to receive said male locking member so as to removably lock said first and second leg members of said surgical clip in said closed position.

11. A polymeric non-snag, low profile surgical clip comprising:
(a) a first leg and a second leg, each of said legs having an inner vessel-clamping surface, an outer surface and a pair of opposing side surfaces flanking said inner and outer surfaces, said inner surfaces being positioned in opposition to each other;
(b) a flexible hinge section integrally disposed between and joining the proximal ends of said first and second legs;
(c) a female locking member positioned on the distal end of said first leg and a male locking member positioned on the distal end of said second leg, said female and male locking members being formed whereby when said first and second leg members are moved from an open position to a closed position about said hinge section, said male locking member is lockingly engaged in said female locking member so as to removably lock said first and second leg members of said surgical clip in said closed position; and
(d) a first protuberance located in the outer surface of said first leg between said hinge section and the distal end of said first leg, and a second protuberance located on the outer surface of said second leg adjacent the distal end of said second leg, said first and second protuberances serving to allow for engagement and application of the surgical clip by a clip applier apparatus.

12. The surgical clip of claim 11, wherein said inner surface of said first leg has a concave radius of curvature between said hinge section and its distal end, said outer surface of said first leg has a convex radius of curvature between said hinge section and its distal end, said inner surface of said second leg has a convex radius of curvature between said hinge section and its distal end, and said outer surface of said second leg has a concave radius of curvature between said hinge section and its distal end.

13. The surgical clip of claim 11, wherein said hinge section has a continuous concave inner surface and a continuous convex outer surface.

14. The surgical clip of claim 11, wherein said female locking member comprises a resilient inwardly turned hook curved toward said second leg member.

15. The surgical clip of claim 11, wherein said male locking member is complementary to said hook of said female locking member whereby when said first and second leg members are moved from an open position to a closed position about said hinge section, said resilient hook of said female locking member contacts said male locking member and is urged open to receive said male locking member so as to removably lock said first and second leg members of said surgical clip in said closed position.

16. A polymeric non-snag, low profile surgical clip comprising:
(a) a first leg and a second leg, each of said legs having an inner vessel-clamping surface, an outer surface and a pair of opposing side surfaces flanking said inner and outer surfaces, said inner surfaces being positioned in opposition to each other;
(b) a flexible hinge section integrally disposed between and joining the proximal ends of said first and second legs;
(c) a female locking member positioned on the distal end of said first leg and a male locking member positioned on the distal end of said second leg, said female and male locking members being formed whereby when said first and second leg members are moved from an open position to a closed position about said hinge section, said male locking member is lockingly engaged in said female locking member so as to removably lock said first and second leg members of said surgical clip in said closed position; and
(d) a first pair of arcuate protuberances joined to opposite sides of said first leg between said hinge section and the distal end of said first leg, and a second pair of arcuate protuberances joined to opposite sides of said second leg adjacent the distal end of said second leg, said first and second pair of arcuate protuberances serving to allow for engagement and application of the surgical clip by a clip applier apparatus.

17. The surgical clip of claim 16, wherein said inner surface of said first leg has a concave radius of curvature between said hinge section and its distal end, said outer surface of said first leg has a convex radius of curvature between said hinge section and its distal end, said inner surface of said second leg has a convex radius of curvature between said hinge section and its distal end, and said outer surface of said second leg has a concave radius of curvature between said hinge section and its distal end.

18. The surgical clip of claim 16, wherein said hinge section has a continuous concave inner surface and a continuous convex outer surface.

19. The surgical clip of claim 16, wherein said female locking member comprises a resilient inwardly turned hook curved toward said second leg member.

20. The surgical clip of claim 19, wherein said male locking member is complementary to said hook of said female locking member whereby when said first and second leg members are moved from an open position to a closed position about said hinge section, said resilient hook of said female locking member contacts said male locking member and is urged open to receive said male locking member so as to removably lock said first and second leg members of said surgical clip in said closed position.
